(19) United States
(12) Patent Application Publication
(10) Pub. No.: US 2006/0241570 A1
(43) Pub. Date: Oct. 26, 2006

(54) Intra-abdominal medical method

(75) Inventor: Peter J. Wilk, New York, NY (US)

Correspondence Address: COLEMAN SUDOL SAPONE, P.C., 714 COLORADO AVENUE, BRIDGE PORT, CT, 06605-1601, US

(73) Assignee: WILK PATENT, LLC, New York, NY (US)

(21) Appl. No.: 11/388,615

(22) Filed: Mar. 24, 2006

Related U.S. Application Data
(60) Provisional application No. 60/674,010, filed on Apr. 22, 2005.

(51) Int. Cl. A61B 1/00; A61B 17/00

(52) U.S. Cl. 606/1; 600/101

(57) ABSTRACT

An endoscopic instrument is inserted into a patient's body to obtain a video image of internal body tissues in an internal space inside the patient's body, a signal encoding the image being transmitted over an electromagnetic signaling link to a remote location beyond a range of direct manual contact with the patient's body and the endoscopic instrument. An artificial opening or perforation is formed in a wall of a hollow internal organ of the patient, the organ communicating with a natural body opening, and a distal end portion of a surgical instrument is inserted through the natural body opening, through the organ and the artificial opening or perforation in the organ wall, and into the internal space. In response to actuator control signals from the remote location via the electromagnetic signaling link, the surgical instrument is automatically actuated to effect a surgical operation on the internal body tissues.

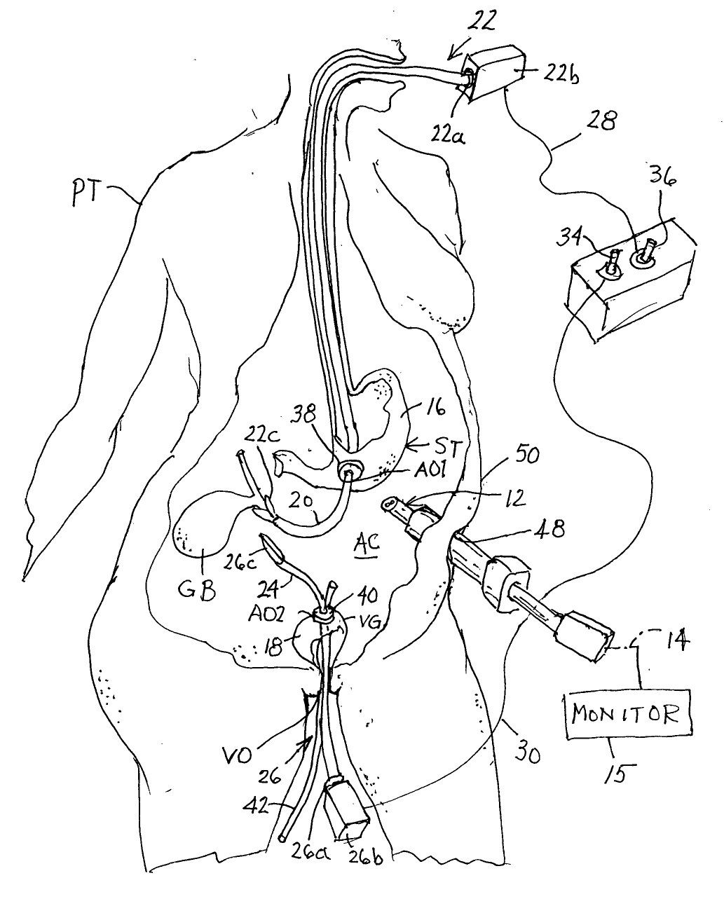

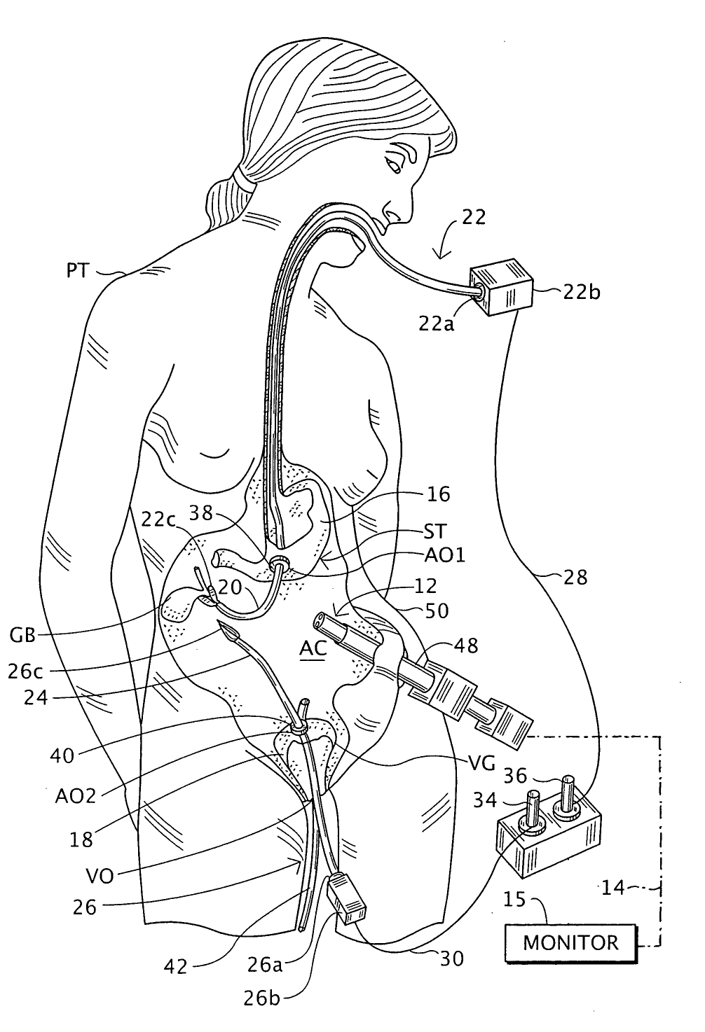

### CROSS-REFERENCE TO RELATED APPLICATION

This application claims the benefit of U.S. Provisional Patent Application No. 60/674,010 filed Apr. 22, 2005.

### BACKGROUND OF THE INVENTION

This invention relates to medical procedures carried out without the formation of an incision in a skin surface of the patient.

Such procedures are described in U.S. Pat. Nos. 5,297,536 and 5,458,131.

As described in those patents, a method for use in intra-abdominal surgery comprises the steps of (a) inserting an incising instrument with an elongate shaft through a natural body opening into a natural body cavity of a patient, (b) manipulating the incising instrument from outside the patient to form a perforation in an internal wall of the natural internal body cavity, and (c) inserting a distal end of an elongate surgical instrument through the natural body opening, the natural body cavity and the perforation into an abdominal cavity of the patient upon formation of the perforation. Further steps of the method include (d) inserting a distal end of an endoscope into the abdominal cavity, (e) operating the surgical instrument to perform a surgical operation on an organ in the abdominal cavity, (f) viewing the surgical operation via the endoscope, (g) withdrawing the surgical instrument and the endoscope from the abdominal cavity upon completion of the surgical operation, and (h) closing the perforation.

Visual feedback may be obtained as to position of a distal end of the incising instrument prior to the manipulating thereof to form the perforation. That visual feedback may be obtained via the endoscope or, alternatively, via radiographic or X-ray equipment.

The abdominal cavity may be insufflated prior to the insertion of the distal end of the endoscope into the abdominal cavity. Insufflation may be implemented via a Veress needle inserted through the abdominal wall or through another perforation in the internal wall of the natural body cavity. That other perforation is formed by the Veress needle itself. U.S. Pat. No. 5,209,721 discloses a Veress needle that utilizes ultrasound to detect the presence of an organ along an inner surface of the abdominal wall.

A method in accordance with the disclosures of U.S. Pat. Nos. 5,297,536 and 5,458,131 comprises the steps of (i) inserting an endoscope through a natural body opening into a natural body cavity of a patient, (ii) inserting an endoscopic type incising instrument through the natural body opening into the natural body cavity, (iii) manipulating the incising instrument from outside the patient to form a perforation in an internal wall of the natural internal body cavity, (iv) moving a distal end of the endoscope through the perforation, (v) using the endoscope to visually inspect internal body tissues in an abdominal cavity of the patient, (vi) inserting a distal end of an elongate surgical instrument into the abdominal cavity of the patient, (vii) executing a surgical operation on the internal body tissues by manipulating the surgical instrument from outside the patient, (viii) upon completion of the surgical operation, withdrawing the surgical instrument and the endoscope from the abdominal cavity, (ix) closing the perforation, and (x) withdrawing the endoscope from the natural body cavity.

The surgical procedures of U.S. Pat. Nos. 5,297,536 and 5,458,131 reduce trauma to the individual even more than laparoscopic procedures. Hospital convalescence stays are even shorter. There would be some advantage to making the surgical procedures of U.S. Pat. Nos. 5,297,536 and 5,458,131 applicable in cases where minute control and mechanical precision are required or where the requisite skill is not available in the vicinity of the patient.

The above-described surgical procedures may be termed “trans-organ” procedures since one passes instruments through an organ in order to reach a target operative site beyond the organ.

### OBJECTS OF THE INVENTION

It is an object of the present invention to provide improvements on the afore-described trans-organ surgical procedures.

It is another object of the present invention to provide a method and/or an associated apparatus that increases the range or applications of trans-organ surgery.

These and other objects of the present invention will be apparent from the drawings and detailed descriptions herein. While every object of the invention is believed to be attained in at least one embodiment of the invention, there is not necessarily any single embodiment that achieves all of the objects of the invention.

### SUMMARY OF THE INVENTION

A surgical kit in accordance with the present invention comprises a plurality of surgical instruments each having an elongate flexible shaft adapted for insertion through a natural body opening and through a perforation in an internal organ into a body cavity. The kit additionally comprises a plurality of coupling elements each disposed at a proximal end of the elongate flexible shaft of a respective one of the surgical instruments for operatively connecting such instrument to a respective servo-mechanism device responsive to control signals carried by an electromagnetic signaling link. In addition, a plurality of port elements deployable in walls of internal organs is provided for enabling a passage of the distal end portions of the instruments.

The surgical kit may further comprising an elongate flexible tube for introducing pressurized gas into an internal space of a patient, the flexible tube being connected to one of the port devices so as to extend from one side of such port device to an opposite side thereof.

A surgical method that may utilize the kit of the invention comprises (1) inserting an endoscopic instrument into a patient's body, (2) obtaining a video image of internal body tissues in an internal space inside the patient's body via the endoscopic instrument, (3) transmitting, over an electromagnetic signaling link, a video signal encoding the video image to a remote location beyond a range of direct manual contact with the patient's body and the endoscopic instrument, (4) forming an artificial opening or perforation in a wall of a hollow internal organ of the patient, the organ communicating with a natural body opening, (5) inserting a distal end portion of a surgical instrument (e.g., from the kit) through the natural body opening, through the organ and the artificial opening or perforation in the organ wall, and into the internal space, (6) receiving actuator control signals from the remote location via the electromagnetic signaling link, and (7) automatically operating the inserted surgical instrument in response to the received actuator control signals to effect a surgical operation on the internal body tissues.

The method may further comprise attaching a port device (e.g., from the surgical kit) to the organ wall at the artificial opening or perforation, so that the surgical instrument is inserted through an opening or aperture in the port device.

Pressurized gas may be introduced into the internal space via an elongate tube (e.g., from the kit) communicating with the internal space via the port device.

The method may additionally comprise the steps of receiving additional control signals from the remote location via the electromagnetic signaling link and automatically operating the endoscopic instrument in response to the additional signals. The endoscopic instrument may be operated to vary the video image. More specifically, the endoscopic instrument may be mechanically moved with respect to the patient's body, thereby varying the video image.

The automatic operation of the surgical instrument(s) may include mechanically moving the surgical instrument(s) with respect to the patient's body and with respect to the endoscopic instrument. More specifically, the surgical instrument may be pivoted and/or translated with respect to the patient's body and with respect to the endoscopic instrument.

The endoscopic instrument may include a flexible insertion member. The inserting of the endoscopic instrument then includes passing the insertion member through an incision or perforation formed in a wall of a hollow internal organ of the patient communicating with a natural body opening. Alternatively, the endoscopic instrument includes a laparoscope which is inserted through the abdominal wall. In any event, operating in the abdominal cavity advantageously includes an inflating of that internal space.

### BRIEF DESCRIPTION OF THE DRAWING

The sole FIGURE of the drawing is a schematic view of person's abdominal region, showing a remotely controlled operating system utilized in the performance of a trans-organ surgical procedure.

### DETAILED DESCRIPTION

As illustrated in the drawing, a trans-organ procedure in accordance with the teachings of U.S. Pat. Nos. 5,297,536 and 5,458,131 includes inserting an endoscopic instrument 12 (rigid or flexible endoscope) into a patient's body PT either laparoscopically as illustrated or via a hollow internal organ such as the stomach, vagina, colon, or urinary bladder. The endoscopic instrument 12 is operated to obtain a video image of internal body tissues in an internal space such as the abdominal cavity AC inside the patient's body. The internal body tissues may include the stomach ST, the gall bladder GB, and the vagina or colon VG. A video signal encoding the video image is transmitted from endoscope 12 over an electromagnetic signaling link 14 to a video monitor 15 at a remote location beyond a range of direct manual contact with the patient's body PT and the endoscopic instrument 12.

Pursuant to the teachings of teachings of U.S. Pat. Nos. 5,297,536 and 5,458,131, an artificial opening or perforation AO1 is formed in a wall 16 of a hollow internal organ such as the stomach ST, which communicates with the mouth (not shown), a natural body opening. Another artificial opening or perforation AO2 is formed in a wall 18 of a hollow internal organ such as the vagina VG, which communicates with the ambient environment via the vaginal orifice VO.

A distal end portion 20 of an elongate flexible shaft (not separately labeled) of a first surgical instrument 22 is inserted through the patient's mouth (not shown), through the wall 16 of stomach ST and the artificial opening or perforation AO1 in the organ wall 16, and into the abdominal cavity AC. Similarly, a distal end portion 24 of an elongate flexible shaft (not separately labeled) of a second surgical instrument 26 is inserted through the patient's vaginal orifice VO, through the wall 18 of vagina VG and the artificial opening or perforation AO2 in the organ wall 18, and into the abdominal cavity AC.

Pursuant to the teachings of U.S. Pat. Nos. 5,217,003 and 5,217,453, surgical instruments 22 and 26 are operatively connected via electromagnetic signaling links 28 and 30 (wired or wireless; across-the-room or intercontinental, for example) to a remote control panel 32 having manual actuators 34 and 36 for enabling a remote user (surgeon) to control the movements and operative activities of instruments 22 and 26. For example, actuators 34 and 36 control the orientations of the distal end portions 20 and 24 during an insertion procedure and a subsequent operation. Signals transmitted from the remote control panel 32 via the electromagnetic signaling links 28 and 30 result in an automatic operation of the surgical instruments 22 and 26 to effect a surgical operation on the internal body tissues.

To that end, instruments 22 and 26 are provided at the proximal ends of their respective elongate flexible shafts with coupling elements 22a and 26a for operatively connecting the instrument shafts to servomechanisms 22b and 26b. Servomechanisms 22b and 26b function to translate the instruments 22 and 26 alternately into and out of the patient PT, to steer the distal ends 20 and 24 of the instruments, and to actuate operative tips 22c and 26c of the instruments.

Accordingly, the robotic surgery teachings of U.S. Pat. Nos. 5,217,003 and 5,217,453 are followed to control, manipulate, and operate the endoscopic surgical instruments in the trans-organ procedures of U.S. Pat. Nos. 5,297,536 and 5,458,131.

As described in U.S. Pat. Nos. 5,297,536 and 5,458,131, port devices 38 and 40 are attached to organ walls 16 and 18 at artificial openings or perforations AO1 and AO2, so that surgical instruments 22 and 26 may pass through stomach ST and vagina VG on their way to a surgical site inside abdominal cavity AC. Port devices 38 and 40 may take any form that enables a passage of instruments 22 and 26 through the walls of stomach ST and vagina VG into the abdominal cavity AC. Typically, port devices 38 and 40 are made of flexible polymeric film material that enables a passage of each port device in a collapsed configuration into stomach ST or vagina VG. There the port devices are unfolded to assume expanded use configurations. Concomitantly, port devices 38 and 40 may each comprise a disk and a balloon (not shown) or two balloon elements (not shown) that inflate to compress or clamp the respective organ wall during use of the port device.

Pursuant to another feature of the present invention, pressurized gas may be introduced into the abdominal cavity AC via an elongate tube 42 communicating with the abdominal cavity via a port device 38 or 40.

The remote control panel 32 may issue additional control signals via an electromagnetic signaling link 44 connected to endoscopic optical instrument 12 for automatically operating the endoscopic instrument 12 to vary the video image transmitted to the monitor 15 at the remote location. More specifically, the endoscopic instrument 12 may be mechanically moved with respect to the patient's body PT, thereby varying the video image.

Surgical instruments 22 and 26 and particular distal end portions 20 and 24 thereof may be mechanically moved with respect to the patient's body PT and with respect to the endoscopic instrument 12. More specifically, the distal end portions 20 and 24 of surgical instruments 22 and 26 may be pivoted and/or translated with respect to the patient's body PT and with respect to the endoscopic instrument 12.

Endoscopic instrument 12 includes a flexible insertion member 46 passed through an incision or perforation 48 formed in an abdominal wall 50 of the patient or of a hollow internal organ such as the stomach, vagina, colon, or urinary bladder communicating with a respective natural body opening.

Surgical instruments 22 and 26, as well as connectors to actuators 34 and 36 and port devices 38 and 40, may be marketed collectively in the form of surgical kits, where the instruments 22, 26, etc., are designed for carrying out respective intra-abdominal surgical procedures via trans-organ pathways.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are profferred by way of example to facilitate comprehension of the invention and should not be construed to limit the scope thereof.

What is claimed is:

1. A surgical method comprising:
inserting an endoscopic instrument into a patient's body;
obtaining a video image of internal body tissues in an internal space inside said patient's body via said endoscopic instrument;
transmitting, over an electromagnetic signaling link, a video signal encoding said video image to a remote location beyond a range of direct manual contact with said patient's body and said endoscopic instrument;
forming an artificial opening or perforation in a wall of a hollow internal organ of the patient, said organ communicating with a natural body opening;
inserting a distal end portion of a surgical instrument through the natural body opening, through said organ and said artificial opening or perforation in the organ wall, and into said internal space;
receiving actuator control signals from said remote location via said electromagnetic signaling link; and
automatically operating said surgical instrument in response to the received actuator control signals to effect a surgical operation on said internal body tissues.

2. The surgical method defined in claim 1, further comprising attaching a port device to said organ wall at said artificial opening or perforation, the inserting of said surgical instrument including inserting a shaft of said surgical instrument through an opening or aperture in said port device.

3. The surgical method defined in claim 2, further comprising introducing pressurized gas into said internal space via an elongate tube communicating with said internal space via said port device.

4. The method recited in claim 1, further comprising the steps of receiving additional control signals from said remote location via said electromagnetic signaling link and automatically operating said endoscopic instrument in response to said additional signals.

5. The method recited in claim 4 wherein said step of automatically operating said endoscopic instrument includes the step of operating said endoscopic instrument to vary said video image.

6. The method recited in claim 5 wherein said step of automatically operating said endoscopic instrument includes the step of mechanically moving said endoscopic instrument with respect to the patient's body, thereby varying said video image.

7. The method recited in claim 1 wherein said step of automatically operating said surgical instrument includes the step of mechanically moving said surgical instrument with respect to the patient's body and with respect to said endoscopic instrument.

8. The method recited in claim 7 wherein said step of mechanically moving said surgical instrument includes the steps of translating said surgical instrument with respect to the patient's body and with respect to said endoscopic instrument.

9. The method recited in claim 1 wherein said endoscopic instrument includes a flexible insertion member and the inserting of said endoscopic instrument includes passing said insertion member through an incision or perforation formed in a wall of a hollow internal organ of the patient communicating with a natural body opening.

10. The method recited in claim 1, further comprising the step of inflating said internal space.

11. A surgical kit comprising: a plurality of surgical instruments each having an elongate flexible shaft adapted for insertion through a natural body opening and through a perforation in an internal organ into a body cavity; a plurality of coupling elements each disposed at a proximal end of the elongate flexible shaft of a respective one of said surgical instruments for operatively connecting such instrument to a respective servo-mechanism device responsive to control signals carried by an electromagnetic signaling link; and a plurality of port elements deployable in walls of internal organs for enabling a passage of the distal end portions of said instruments.

12. The surgical kit defined in claim 11, further comprising an elongate flexible tube for introducing pressurized gas into an internal space of a patient, said flexible tube being connected to one of said port devices so as to extend from one side of said one of said port devices to an opposite side thereof.
